(19) United States
(12) Patent Application Publication
(10) Pub. No.: US 2026/0305946 A1
(43) Pub. Date: Oct. 8, 2026

(54) SYSTEMS AND METHODS FOR CARDIOVASCULAR MAGNETIC RESONANCE IMAGING

(71) Applicant: Ohio State Innovation Foundation, Columbus, OH (US)

(72) Inventors: Syed Murtaza ARSHAD, Columbus, OH (US); Rizwan AHMAD, Hilliard, OH (US)

(21) Appl. No.: 19/483,271

(22) PCT Filed: May 13, 2024
(86) PCT No.: PCT/US2024/029098

§ 371 Date: Nov. 11, 2025

Related U.S. Application Data
(60) Provisional application No. 63/466,088, filed on May 12, 2023.

(51) Int. Cl. A61B 5/00; A61B 5/026; A61B 5/055; G01R 33/563; G01R 33/565; G01R 33/567

(52) U.S. Cl. CPC A61B 5/0044; A61B 5/0263; A61B 5/055; A61B 5/7207; A61B 5/7289; G01R 33/56308; G01R 33/56509; G01R 33/5676; A61B 2576/023

(57) ABSTRACT

An example computer implemented method for image reconstruction is described herein. The example method includes receiving k-space data from an magnetic resonance imaging (MRI) machine; sorting the k-space data into a plurality of bins, each of the plurality of bins corresponding to a respective phase of a respiratory cycle; identifying a plurality of outliers in the k-space data; and reconstructing an image from the k-space data using a compressive sensing (CS) technique configured to remove the plurality of outliers from the k-space data, where the CS technique is configured to apply structured sparsity regularization to the plurality of outliers in the k-space data.

### CROSS-REFERENCE TO RELATED APPLICATIONS

This application claims the benefit of U.S. provisional patent application No. 63/466,088 filed on May 12, 2023, and titled “SYSTEMS AND METHODS FOR CARDIOVASCULAR MAGNETIC RESONANCE IMAGING,” the disclosure of which is expressly incorporated herein by reference in its entirety.

### STATEMENT REGARDING FEDERALLY FUNDED RESEARCH

This invention was made with government support under grant/contract no. HL151697 awarded by the National Institutes of Health. The government has certain rights in the invention.

### BACKGROUND

Magnetic Resonance Imaging (MRI) is a non-invasive imaging modality that produces images of soft tissue structures from inside the body, such as organs and vessels, with visible contrast resolution. Cardiovascular Magnetic Resonance (CMR) is a robust MRI technique used to image heart and its chambers, blood vessels, and surrounding tissues. CMR has become an essential tool for evaluating heart structure and function and for diagnosis of cardiovascular diseases.

Improvements to MRI and CMR can improve imaging and ultimately the diagnosis and treatment of various diseases.

### SUMMARY

In some aspects, implementations of the present disclosure include a computer-implemented method for image reconstruction, the method including: receiving k-space data from an magnetic resonance imaging (MRI) machine; sorting the k-space data into a plurality of bins, each of the plurality of bins corresponding to a respective phase of a respiratory cycle; identifying a plurality of outliers in the k-space data; and reconstructing an image from the k-space data using a compressive sensing (CS) technique configured to remove the plurality of outliers from the k-space data, wherein the CS technique is configured to apply structured sparsity regularization to the plurality of outliers in the k-space data.

In some aspects, implementations of the present disclosure include a computer-implemented method, wherein the CS technique includes a data fidelity term and a regularization term, wherein the regularization term applies the structured sparsity regularization to the plurality of outliers in the k-space data.

In some aspects, implementations of the present disclosure include a computer-implemented method, wherein the CS technique is performed according to

$$\hat{x} = arg {min}_{x , v} \{ \frac{1}{2} {‖ Ax - ( y - v ) ‖}_{2}^{2} + {λ}_{1} {‖ Wx ‖}_{1} + {λ}_{2} {‖ v ‖}_{2 , 1} \} , where \hat{x} = Reconstructed Image y = Collected K Space Data A = Forward Operator W = Sparsifying ( NDW ) Transform v = Rejected Outlier data points and {λ}_{i} = Tuning Parameters .$$

In some aspects, implementations of the present disclosure include a computer-implemented method, wherein identifying the plurality of outliers in the k-space data includes identifying one or more outliers in the k-space data in each of the plurality of bins.

In some aspects, implementations of the present disclosure include a computer-implemented method, wherein the reconstructed image is a motion compensated image.

In some aspects, implementations of the present disclosure include a computer-implemented method, wherein the k-space data includes cardiac MRI data.

In some aspects, implementations of the present disclosure include a computer-implemented method, wherein the reconstructed image includes a measure of net flow or peak velocity in a heart.

In some aspects, implementations of the present disclosure include a system for MRI imaging, the system including: an MRI machine; and a controller in operable communication with the MRI machine, wherein the controller includes a processor and a memory, the memory having computer-executable instructions stored thereon that, when executed by the processor, cause the processor to: receive k-space data from the MRI machine; sort the k-space data into a plurality of bins, each of the plurality of bins corresponding to a respective phase of a respiratory cycle; identify a plurality of outliers in the k-space data; and reconstruct an image from the k-space data using a compressive sensing (CS) technique configured to remove the plurality of outliers from the k-space data, wherein the CS technique is configured to apply structured sparsity regularization to the plurality of outliers in the k-space data.

In some aspects, implementations of the present disclosure include a system, wherein the MRI machine is configured to perform cardiovascular magnetic resonance imaging.

In some aspects, implementations of the present disclosure include a system, wherein the CS technique includes a data fidelity term and a regularization term, wherein the regularization term applies the structured sparsity regularization to the plurality of outliers in the k-space data.

In some aspects, implementations of the present disclosure include a system, wherein the CS technique is performed according to

$$\hat{x} = arg {min}_{x , v} \{ \frac{1}{2} {‖ Ax - ( y - v ) ‖}_{2}^{2} + {λ}_{1} {‖ Wx ‖}_{1} + {λ}_{2} {‖ v ‖}_{2 , 1} \} , where \hat{x} = Reconstructed Image y = Collected K Space Data A = Forward Operator W = Sparsifying ( NDW ) Transform v = Rejected Outlier data points and {λ}_{i} = Tuning Parameters .$$

In some aspects, implementations of the present disclosure include a system, wherein identifying the plurality of outliers in the k-space data includes identifying one or more outliers in the k-space data in each of the plurality of bins.

In some aspects, implementations of the present disclosure include a system, wherein the reconstructed image is a motion compensated image.

In some aspects, implementations of the present disclosure include a system, wherein the k-space data includes cardiac MRI data.

In some aspects, implementations of the present disclosure include a system, wherein the reconstructed image includes a measure of net flow or peak velocity in a heart.

In some aspects, implementations of the present disclosure include a computer-readable medium having instructions stored therein, wherein execution of the instructions by a processor, causes the processor to: receive k-space data from an magnetic resonance imaging (MRI) machine; sort the k-space data into a plurality of bins, each of the plurality of bins corresponding to a respective phase of a respiratory cycle; identify a plurality of outliers in the k-space data; and reconstruct an image from the k-space data using a compressive sensing (CS) technique configured to remove the plurality of outliers from the k-space data, wherein the CS technique is configured to apply structured sparsity regularization to the plurality of outliers in the k-space data.

In some aspects, implementations of the present disclosure include a computer-readable medium, wherein the CS technique includes a data fidelity term and a regularization term, wherein the regularization term applies the structured sparsity regularization to the plurality of outliers in the k-space data.

In some aspects, implementations of the present disclosure include a computer-readable medium, wherein the CS technique is performed according

$$\hat{x} = arg {min}_{x , v} \{ \frac{1}{2} {‖ Ax - ( y - v ) ‖}_{2}^{2} + {λ}_{1} {‖ Wx ‖}_{1} + {λ}_{2} {‖ v ‖}_{2 , 1} \} , where \hat{x} = Reconstructed Image y = Collected K Space Data A = Forward Operator W = Sparsifying ( NDW ) Transform v = Rejected Outlier data points and {λ}_{i} = Tuning Parameters .$$

In some aspects, implementations of the present disclosure include a computer-readable medium, wherein identifying the plurality of outliers in the k-space data includes identifying one or more outliers in the k-space data in each of the plurality of bins.

In some aspects, implementations of the present disclosure include a computer-readable medium, wherein the reconstructed image is a motion compensated image including a measure of net flow or peak velocity in a heart.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 illustrates a flow chart of an example method for image reconstruction, according to implementations of the present disclosure.

FIG. 2 illustrates an example system for performing image reconstruction in cardiac magnetic resonance imaging, according to implementations of the present disclosure.

FIG. 3 is an example computing device.

FIG. 4 illustrates an example of self-gating on example waveforms.

FIG. 5 illustrates study results using an example implementation of the present disclosure.

FIG. 6 illustrates study results using an example implementation of the present disclosure.

FIG. 7A illustrates a visual comparison of a representative frame from two different datasets reconstructed using compressed sensing (CS) and an example implementation of the present disclosure.

FIG. 7B illustrates example x-t profiles based on the frames shown in FIG. 7A.

FIG. 8A illustrates a qualitative comparison of CS and an example implementation of the present disclosure using representative aortic flow profiles, magnitude and velocity components of rest 4D flow images.

FIG. 8B illustrates representative magnitude image, velocity component in the superior-inferior direction (vₓ), velocity component in the right-left direction (v_{y}), and velocity component in the anterior-posterior direction (v_{z}) from CS and CORe for the study results shown in FIG. 8A.

FIG. 9A illustrates a qualitative comparison of CS and CORe reconstructions using representative aortic flow profiles, magnitude and velocity components of stress 4D flow images,

FIG. 9B illustrates representative magnitude image, velocity component in the superior-inferior direction (vₓ), velocity component in the right-left direction (v_{y}), and velocity component in the anterior-posterior direction (v_{z}) from CS and CORe are shown at systole (peak flow) for the 2D plane 1 for the example comparison shown in FIG. 9A.

FIG. 10 illustrates example imaging parameters for 2D-PC, 3D Cine, 4D flow at rest, and 4D flow during exercise studies, where the ranges indicate minimum and maximum values, according to implementations of the present disclosure.

FIG. 11 illustrates example NMSE (dB) and SSIM results derived from 55 random draws in studies of an example implementation of the present disclosure.

FIG. 12 illustrates results of a blinded reader study conducted on seven 3D cine datasets where each score represents an average±SD from three CMR expert readers.

FIG. 13 illustrates a comparison of net flow (ml/beat) and peak velocity (cm/s) in a study of an example implementation of the present disclosure at the Aao plane shown in FIG. 8A.

FIG. 14 illustrates a comparison of flow quantification from CS and CORe reconstructions of stress 4D flow the values represent the mean±SD net flow across Aao and Dao planes, as defined in FIG. 9A. Bold values indicate the lower standard deviation. The last row indicates the average standard deviation across Aao and Dao in CS and CORe reconstructions.

FIG. 15 illustrates an illustration of outliers in a readout, according to implementations of the present disclosure.

FIG. 16 illustrates a comparison of outliers identified by SO to outliers identified by CORe, according to an example implementation of the present disclosure.

FIG. 17A illustrates cardiac and respiratory signals from a rest 4D flow dataset, according to an example implementation of the present disclosure.

FIG. 17B illustrates cardiac and respiratory motion signals obtained using a stress 4D flow dataset, according to an example implementation of the present disclosure.

FIG. 18 illustrates an example alternating direction method of multipliers optimization method (ADMM), according to implementations of the present disclosure.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for performing reconstruction of cardiovascular magnetic resonance (CMR) images, it will become evident to those skilled in the art that the implementations are not limited thereto, but are applicable for reconstructing other types of images.

Implementations of the present disclosure include improvements to image processing and analysis for cardiac magnetic resonance imaging (“CMR”). CMR can have significant benefits, including providing a comprehensive assessment of the cardiovascular structure, function, and morphology. CMR-based assessment is unmatched in assessing cardiac function, detecting myocardial fibrosis, and providing comprehensive tissue characterization. [1]. Additionally, magnetic resonance imaging (MRI) is a versatile imaging modality that provides high soft-tissue contrast without ionizing radiation.

Existing CMR implementations can have significant limitations caused by motion. “Bulk motion” caused by movement of the patient in an MRI system can impact the accuracy of CMR images. Additionally, CMR images are affected by both cardiac and respiratory motion. Accordingly, CMR images can be degraded by at least three types of motion that occur while the CMR image is being captured. Clinical CMR can use breath-held segmented acquisition, where the data collection is synchronized with cardiac activity recorded using an electrocardiogram (ECG). [2,3] This approach may not be feasible for subjects who cannot hold their breath or have arrhythmias. 2D free-breathing realtime imaging can be used as an alternative, and may not require breath-holding or a regular cardiac rhythm. [4, 5]. However, due to a 6 to 10 mm slice thickness and difficulty in temporally aligning different slices, 2D imaging does not yield a true 3D visualization of heart anatomy. Free-breathing volumetric CMR circumvents the limitations of 2D imaging, but the respiratory motion of the heart in free-breathing acquisition remains a major challenge. [6]. Free-breathing volumetric CMR performed under ECG guidance and prospective respiratory gating using navigator echoes has been proposed and validated for many CMR applications. However, depending on the breathing pattern and the extent of arrhythmia, this approach may lead to unpredictably long acquisition times. Also, navigator echoes disrupt the steady-state of magnetization and thus are not compatible with several common CMR pulse sequences. More recently, there has been renewed interest in free-running volumetric imaging (FRV), where data are collected continuously for several minutes without guidance from navigator echoes, respiratory bellows,⁷ or even ECG.⁸ FRV provides an easier clinical setup with minimal planning and the added flexibility of determining the number of cardiac and respiratory bins at the time of reconstruction.

FRV methods can include self-gating, [9] where a fixed segment of k-space-typically a readout in the inferior-superior direction-is traversed periodically during the acquisition. The dynamic changes in self-gating data segments are attributed to physiological motions. A common approach for extracting respiratory and cardiac signals relies on performing blind source separation by a combination of band-pass filtering, principal component analysis (PCA), and independent component analysis (ICA). [8]. These motion signals are then employed to sort k-space data into multiple respiratory and cardiac motion bins. The data binning is then followed by image reconstruction in the spatial-cardiacrespiratory domain, leading to “motion-resolved” imaging. [10, 11]. An alternative to motion-resolved imaging is to integrate a respiratory motion model into the reconstruction framework to map images at different respiratory states to a target (e.g., expiratory) state. [12, 13]. This approach does not resolve the respiratory dimension but utilizes all the data to improve the image quality of the target respiratory state. However, the efficacy of these FRV imaging methods depends on the quality of extracted motion signals.

Accurately extracting cardiac and respiratory signals from the self-gating data is a challenging problem, especially when heart-rate variability, arrhythmia, or inconsistent breathing patterns are present. Although Pilot Tone (PT) provides an alternative to self-gating, [14] the extraction of physiological motion from PT faces similar challenges. [15]. Any inaccuracy in the extraction of physiological motion signals would result in incorrect assignment of k-space lines to cardiac and respiratory bins, resulting in image artifacts. In the case of in-magnet exercise stress CMR, the problem of reliably extracting respiratory and cardiac signals becomes even more challenging due to the movement of the torso. [16]

Implementations of the present disclosure (e.g., the example implementation referred to herein as “Compressive recovery with Outlier Rejection (CORe)) can overcome these limitations. In contrast to compressed sensing (CS), [17] implementations of the present disclosure can provide more robust reconstruction by suppressing the outliers, which are invariably present in FRV acquisitions due to imperfect binning. CORe explicitly models the outliers using an additive auxiliary variable, which can be jointly estimated with the image. Unlike previous work in image inpainting where such an auxiliary variable is assumed to be pixel-wise sparse, [18] implementations of the present disclosure can leverage the structure in the MRI data to impose sparsity at a group (readout) level. Additionally, studies described herein implement CORe using the alternating direction method of multipliers (ADMM) optimization algorithm and apply it to large-scale imaging problems. Examples described herein include validation and testing of implementations of the present disclosure using two simulation studies as well as data from free-running 3D cine, 4D flow, and exercise stress 4D flow imaging.

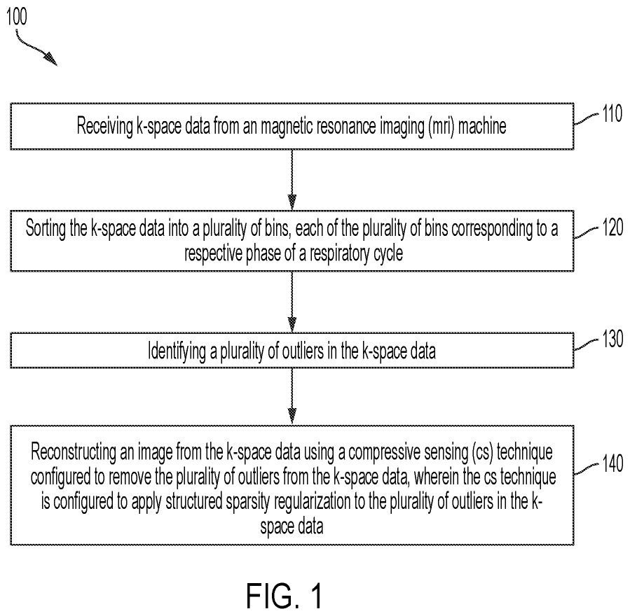

Described herein are systems and methods reconstructing images. With reference to FIG. 1, an example method 100 of image reconstruction is shown. The example method 100 includes receiving k-space data from a magnetic resonance imaging (MRI) machine at step 110. Optionally, the k-space data can include cardiac MRI data.

The example method 100 further includes sorting the k-space data into a plurality of bins, at step 120, where each of the plurality of bins corresponding to a respective phase of a respiratory cycle.

The example method 100 further includes identifying outliers in the k-space data at step 130. Optionally, identifying the plurality of outliers in the k-space data can include identifying one or more outliers in the k-space data in each of the plurality of bins.

At step 140, the example method 100 can further include reconstructing an image from the k-space data using a compressive sensing (CS) technique configured to remove the plurality of outliers from the k-space data, where the CS technique is configured to apply structured sparsity regularization to the plurality of outliers in the k-space data. Optionally, the CS technique can include a data fidelity term and a regularization term, wherein the regularization term applies structured sparsity regularization to the plurality of outliers in the k-space data.

In some implementations, the CS technique can be performed according to according to

$$\hat{x} = arg {min}_{x , v} \{ \frac{1}{2} {‖ Ax - ( y - v ) ‖}_{2}^{2} + {λ}_{1} {‖ Wx ‖}_{1} + {λ}_{2} {‖ v ‖}_{2 , 1} \} , where \hat{x} = Reconstructed Image y = Collected K Space Data A = Forward Operator W = Sparsifying ( NDW ) Transform v = Rejected Outlier data points and {λ}_{i} = Tuning Parameters .$$

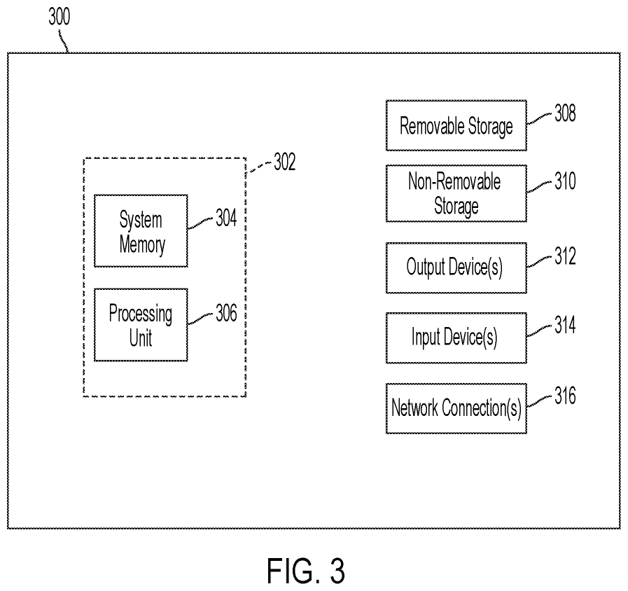

It should be understood that the reconstructed image can be a motion compensated image. An example motion compensated image is a cardiac MRI, which can require motion compensation to image the moving heart. It should also be understood that implementations of the present disclosure can be implemented as computer-readable media, as described with reference to FIG. 3 herein.

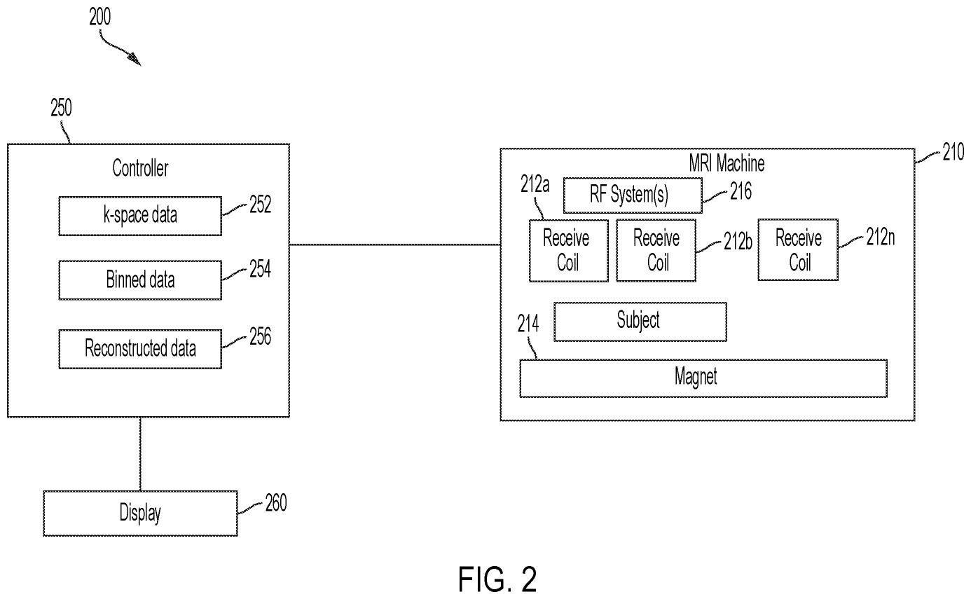

Implementations of the present disclosure include systems that can be used to improve the performance of MRI systems, including Cardiac MRI systems. FIG. 2 illustrates an example system 200, according to implementations of the present disclosure. The system 200 includes an MRI machine 210, which is optionally a cardiac MRI (CMR) machine. The MRI machine 210 includes a number of receive coils 212a-212n that acquire MRI data. The receive coils 212a-212n can be configured for receiving signals form a specific body part (e.g., Cardiac MR signals). The MRI machine 210 also includes a magnet 214 to polarize the subject of the MRI Machine, 210 and/or RF system 216 configured to excite the subject of the MRI machine 210 and generate the signals received by the receive coils 212a-212n. It should be understood that any number and/or configurations of RF systems 216, receive coils 212a-212n, and/or magnets 214 can be used to implement the MRI machine 210, and that the MRI machine 210 is intended only as a non-limiting example. Additionally, it should be understood that the RF systems 216 can optionally include the receive coils 212a-212n. It should also be understood that the RF system 216 can include transmit coils (not shown) and that in some implementations the same coils can be used as transmit coils and receive coils to implement an RF system 216. It should be understood that the subject being measured by the MRI machine can be any portion of an organism or object, for example living and dead animals, humans, plants, etc. as well as industrial samples (e.g. samples of foods, fluid pipes, chemical analysis, etc.)

Still with reference to FIG. 2, the MRI machine 210 can be operably coupled to a controller 250 (e.g., using any kind of wireless or wired network link). The controller 250 can be used to implement any of the methods described herein, for example the methods described with reference to FIG. 1 and/or any of the methods described with reference to Examples 1-3 herein. The controller 250 can be implemented using any or all of the computing device 300 illustrated in FIG. 3. As shown in FIG. 2, the controller 250 can be configured to receive k-space data 252 from the MRI machine 210, as described with reference to step 110 of FIG. 1. The k-space data 252 can represent the 2D/3D Fourier transform of the image measured by the MRI machine 210.

The controller 250 can further be configured to sort the k-space data 252 into binned data 254 as described with reference to step 120 of FIG. 1. Outliers in the binned data 254 can then be removed as described with reference to step 130 of FIG. 1, and the binned data 254 can be used to generate reconstructed data 256.

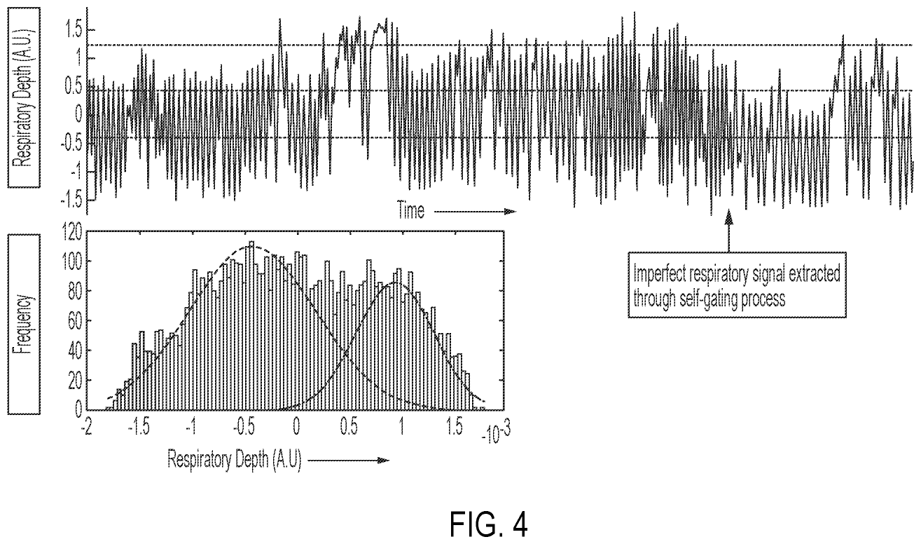

Optionally, the system 200 can further include a display 260. The display 260 can optionally include a separate computing device (e.g., the computing device 300 shown in FIG. 3), and alternatively or additionally can be configured as the same computing device as the controller 250. The display 260 can be configured to output the reconstructed data 256, for example as a motion-compensated CMR image or any other data used in the systems and methods described herein. Example images that can be displayed include images shown in FIGS. 4A-9B, 16, and 17A-17B as described with reference to Examples 1-3, below.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

An example implementation of the present disclosure was designed and tested with MRI scans. MRI scans are relatively long as k-space data is acquired sequentially in the frequency domain, compared to traditional camera imaging where whole pixel data is collected simultaneously. Due to long scan times, volumetric CMR such as 3D MRI or 4D flow can become more challenging as patients cannot hold their breath for such a long time, and respiratory motion creates motion artifacts which significantly reduces the image quality. This can show the need of motion resolved MRI, in which scan is performed in free-breathing state and respiratory motion is resolved using motion compensation techniques such as prospective navigator gating or retrospective self-gating. The accuracy of these respiratory compensation methods relies on the respiratory signal acquired during the scan. For example, in self-gating, k-space data acquired during different phases of the respiratory cycle is sorted into separate bins based on the respiratory signal, known as k-space binning. An example of self-gating is shown in FIG. 4. As a result of the presence of irregularities in breathing patterns, cardiac motion, patient movements, and other sources of noise associated with MRI procedure, it can be almost impossible to extract a perfect respiratory signal. Consequently, bin assignment for a fraction of readouts (outlier encodings) can be incorrect. In the case of exercise stress cardiac MRI, the quality of the self-gating signal degrades even further due to exaggerated breathing patterns, and contamination from exercise-induced torso movement. Therefore, these respiratory compensation methods do not suppress respiratory motion completely, leading to image artifacts.

Implementations of the present disclosure include an outlier rejection scheme in the image reconstruction process. Implementations of the present disclosure can minimize motion artifacts by suppressing contributions from k-space readouts that have been erroneously assigned to the wrong respiratory bin. An example embodiment of the present disclosure, referred to herein as Compressive recovery with Outlier Rejection (CORe), includes a motion robust extension of compressed sensing (CS), which reduces the motion artifacts in the reconstruction process by suppressing the outliers in measured data. Clinically approved products can use Compressed Sensing (CS) as a reconstruction method from undersampled data. CS optimization methods contain a data fidelity and a regularization term.

CORe solves the reconstruction problem from motion-corrupted k-space. CORe applies structured sparsity regularization on outlier encodings, in contrast to L1 norm sparse regularization proposed in Dong et al (2011)*.

A study was performed comparing the example implementation of CORe, CS and method proposed in Dong et al (2011) (Dong, Bin & Ji, Hui & Li, Jia & Shen, Zuowei & Xu, Yuhong. (2011). Wavelet Frame Based Blind Image Inpainting. Applied and Computational Harmonic Analysis. 32. 368-379) for reconstructing a 2D Shepp-Logan phantom from an undersampled k-space data with an acceleration rate at 2.5, containing Gaussian noise and outliers added to 20% of sampled k-space data. This experiment was performed 50 times with random sampling and random placement of outliers in each realization. The example implementation of CORe demonstrated superior performance to CS and the method proposed in Dong et al. (2011)* with average NMSE of −13.5, −10.2−12.15 dBs respectively, and SSIM of 0.98, 0.96, 0.97 respectively.

To demonstrate advantages of the example implementation, another study was performed for the reconstruction of a 2D image originating from a bimodal dynamic phantom (simulating inspiratory and expiratory phases of respiratory motion). To simulate contamination with outliers, approximately 10% of the k-space data is randomly sampled from the inspiratory phase and the rest of the k-space is randomly filled with data from the expiratory phase. This contaminated k-space is used to reconstruct the expiratory phase image. The simulation is repeated 50 times, each with a random realization of the outlier locations in k-space. The acceleration rate was fixed at 3.5. In the dynamic phantom simulation, reconstruction of the expiratory phase using CORe, CS and Dong et al. (2011) (Dong, Bin & Ji, Hui & Li, Jia & Shen, Zuowei & Xu, Yuhong. (2011). Wavelet Frame Based Blind Image Inpainting. Applied and Computational Harmonic Analysis. 32. 368-379) method for 50 realizations yielded average NMSE values (in dB) of −11.35, −8.78 and −10.67 respectively, and average SSIM values of 0.89, 0.80 and 0.73 respectively.

To validate the improvement offered by the example implementation of CORe in volumetric CMR reconstruction, the study reconstructed three free-running, free-breathing, and fully self-gated 3D cine patient datasets acquired on a clinical 3T scanner (MAGNETOM Vida, Siemens Healthcare, Erlangen, Germany) using Cartesian sampling for a fixed acquisition time of 5 minutes, using CORe and CS. The images reconstructed with CORe compared to CS demonstrated significantly fewer artifacts.

To further validate the advantage of CORe over CS, six free-running, free-breathing, and fully self-gated 4D flow datasets were collected for a fixed acquisition time of 5 minutes using cartesian sampling. The datasets were acquired from 3 healthy volunteers (age range, 22-49 years) under different exercise stress conditions using a clinical 3T scanner (MAGNETOM Vida, Siemens Healthcare, Erlangen, Germany) and a cycle ergometer (MR Ergometer Pedal, Lode, The Netherlands). Two datasets were collected from each volunteer during rest state and exercise at 20 W. To assess improvement in blood flow quantification using CORe as compared to CS, blood flow quantification through ascending aorta over a cardiac cycle was performed. The comparison of both magnitude images and flow curves demonstrate that the suggested method CORe is more effective in suppressing motion artifacts, with the reduction in artifacts being more evident under exercise stress.

Owing to the significance of volumetric and flow MRI in diagnosis of congenital heart diseases, heart valve disorders, aortic diseases, pulmonary diseases, cardiac tumors, among others, there is currently a substantial research focus on the development of an efficient motion-resolved CMR reconstruction. Moreover, exercise stress CMR is an emerging area of research as it provides valuable diagnostic and prognostic information for patients with cardiovascular disease which may not be evident at rest. These clinical applications highlight the importance of this innovation. The computation time for both CS and the example CORe reconstruction algorithms is comparable. Although the example implementation was evaluated and applied to volumetric MRI with motion artifacts, it should be understood that implementations of the present disclosure can also be extended to any other MRI modalities to suppress outliers from various sources in k-space data during reconstruction. Since CORe can include an extension of compressed sensing, its performance is equivalent to CS in the absence of outliers, making it a versatile and suitable choice for a wide range of MRI applications.

### Example 2

An example method referred to herein as “Compressive recovery with Outlier Rejection (CORe),” was designed and tested in studies as described herein. The example method models outliers in the measured data as an additive auxiliary variable. Implementations of the present disclosure enforce MR physics-guided group sparsity on the auxiliary variable, and jointly estimate it along with the image using an iterative algorithm. For evaluation, CORe is first compared to traditional compressed sensing (CS), robust regression (RR), and an existing outlier rejection method using two simulation studies. Then, CORe is compared to CS using seven 3D cine, twelve rest 4D flow, and eight stress 4D flow imaging datasets.

The study results show that CORe outperforms CS, RR, and the existing outlier rejection method in terms of normalized mean square error (NMSE) and structural similarity index (SSIM) across 55 different realizations. The expert reader evaluation of 3D cine images demonstrates that CORe is more effective in suppressing artifacts while maintaining or improving image sharpness. Finally, 4D flow images show that CORe yields more reliable and consistent flow measurements, especially in the presence of involuntary subject motion or exercise stress. The study showed that implementations of the present disclosure can suppress motion artifacts in a wide range of free-running CMR applications.

The example method, CORe, integrates outlier rejection into the compressive reconstruction. The results from the simulation studies show that CORe outperforms standard CS and other robust regression models in terms of image quality. The results from the human subject studies demonstrate that CORe is more effective than CS in suppressing motion artifacts due to imperfect binning. CORe's ability to suppress motion artifacts makes it an attractive candidate for free-running volumetric CMR.

### Theory

In MRI, reconstruction involves estimating the underlying image from noisy and potentially undersampled complex-valued k-space measurements. The measured noisy data are related to the image by:

$$y = Ax + ω \qquad \# ( 1 )$$

where x∈ [symbol] ^{N} is an N-pixel image that has been vectorized, y∈ [symbol] ^{M} is the MRI data measured from C receive coils, ω∈ [symbol] ^{M} is circularly symmetric white Gaussian noise with variance σ², and A∈ [symbol] ^{M×N} is a sensing matrix that incorporates pixel-wise multiplication with coil sensitivity maps, 2D or 3D discrete Fourier transform, and k-space undersampling. The model in Equation (1) is also applicable to dynamic imaging where x and y represent vertical concatenations of pixels and k-space data, respectively, from individual frames, and A represents block-diagonal embedding of the sensing matrices from individual frames.

In CS-based reconstruction [19], Equation (1) is often solved using

$${\hat{x}}_{CS} = {arg min}_{x} \frac{1}{{σ}^{2}} {‖ Ax - y ‖}_{2}^{2} + ℛ ( x ) \qquad ( 2 )$$

where [symbol] (x) is a sparsity promoting prior. Common choices of [symbol] (x) in CS-based MRI reconstruction include λ₁∥Ψx∥₁, where Ψ is a linear sparsifying transform, [20] or λ₁ [symbol] (x), where [symbol] (⋅) computes the total variation. [21] From a Bayesian perspective, a regularizer injects prior belief about the underlying image, x. For example, for [symbol] (x)=λ₁∥Ψx∥₁, \hat{x}_{RL} is a maximum a posteriori (MAP) estimate under the sparsity-promoting prior probability density function of p(x)∝exp (−λ₁∥Ψx∥₁).

CS methods enable higher acceleration rates than possible without sparsity-based priors, [17], [22]. More recently, deep learning (DL)-based reconstruction methods have been shown to outperform sparsity-based CS methods. [23-25]. However, almost all of these CS and DL methods are reliant on Equation (1), which, in the absence of motion, is a valid model for MRI measurements.

In the presence of uncompensated motion, e.g., due to imperfect retrospective data binning, the model in Equation (1) is no longer valid. Implementations of the present disclosure can therefore be used to improve on CS and DL methods that rely on Equation 1, and may not be effective when motion is considered. If one were to consider motion-induced outliers to be additive noise with a heavy-tailed Laplacian distribution, then a common remedy is to use robust regression (RR) by replacing the [symbol] ₂-norm with [symbol] ₁-norm in Equation (2),²⁶ leading to the MAP solution:

$${\hat{x}}_{RR} = {arg min}_{x} {λ}_{0} {‖ Ax - y ‖}_{1} + ℛ ( x ) \qquad ( 3 )$$

A different approach to account for motion induced outliers is to model them as an additive perturbation in k-space using an unknown auxiliary variable, v∈ [symbol] ^{M}, i.e.,

$$y = Ax + v + ω \qquad ( 4 )$$

The model in Equation (4) implies that the underlying image x, additive Gaussian noise co, and uncompensated motion v all contribute to the k-space measurements. However, during image reconstruction process, v is not treated as noise but as an auxiliary unknown variable that exhibits structure from the data acquisition and is jointly estimated with x. With both x and v being unknown, the model in Equation (4) is ill-posed even in the presence of [symbol] (x). To mitigate this issue, one could enforce sparsity on v by assuming that its entries are drawn from i.i.d. Laplacian distribution, i.e., p(v)∝exp (−λ₂∥v∥₁). This assumption encourages v to assume a sparse support in k-space, with its non-zero entries acting as outliers. The resulting optimization problem, termed as sparse outlier (SO), assumes the form:

$${\hat{x}}_{SO} = {arg min}_{x , v} \frac{1}{{σ}^{2}} {‖ Ax - y + v ‖}_{2}^{2} + ℛ ( x ) + {λ}_{2} {‖ v ‖}_{1} . \qquad ( 5 )$$

In Equation (5), although both \hat{x}_{SO} and \hat{v} are returned, the latter is ignored for brevity. Under mild assumptions, Equation (5) is a MAP estimate of the model in Equation (4).

The optimization problem in Equation (5) does not fully utilize the data structure specific to MRI measurements [18]. The MRI data are invariably collected in the form of readouts, where a sequence of k-space samples is collected within a short period of time that is of the order of a millisecond. Given that physiological motions occur at much larger time scales, the duration of a single readout can be considered negligible. Therefore, instead of assuming that the motion impacts individual samples in k-space as in Equation (5), the example implementation can assume that the motion impacts an entire readout. This modeling choice is not only more realistic but also more robust, as it is easier to detect an entire readout corrupted by motion compared to individual k-space entries.

To treat all Kk-space samples in each of the J readouts as a group, the example implementation can leverage group sparsity. [27, 28] This can be realized by enforcing a hybrid [symbol] ₂− [symbol] ₁ penalty on v, where the [symbol] ₂ norm is first computed along the readout dimension followed by the [symbol] ₁ norm along all remaining dimensions, leading to the proposed CORe formulation, i.e.,

$${\hat{x}}_{CO} = {arg min}_{x , v} \frac{1}{{σ}^{2}} {‖ Ax - y + v ‖}_{2}^{2} + ℛ ( x ) + {λ}_{2} {‖ g ( v ) ‖}_{1} . \qquad \# ( 6 )$$

In Equation (6), the vector g(v) denotes [symbol] ₂-norms of k-space readouts. With vⱼ∈ [symbol] ^{K×1} representing the j^{th} readout,

$$g ( v ) {=}^{Δ} {[ {\tilde{v}}_{1} , {\tilde{v}}_{2} , ... , {\tilde{v}}_{J} ]}^{T} ∈ {ℝ}^{J \times 1} , \\ where {\tilde{v}}_{j} = {‖ {v}_{j} ‖}_{2} ∈ ℝ and {‖ g ( v ) ‖}_{1} = {\sum }_{j = 1}^{J} \vert {\tilde{v}}_{j} \vert .$$

As previously, although both \hat{x}_{CO} and \hat{v} are returned in Equation (6), the latter is ignored for brevity. The optimization in Equation (6) provides automatic separation of measured data into outlier estimation, \hat{v}, and k-space data, y−\hat{v}, that is used for image reconstruction. Note, CORe may not make assumptions about the nature of the physiological motion, e.g., rigid vs. nonrigid, or the binning process; it can employ an additive auxiliary variable as a means to separate contributions from corrupted readouts that are inconsistent with the rest of the data.

Traditional CS and RR methods require adjusting one regularization parameter, λ₁, which controls the relative emphasis on [symbol] (x) compared to the data fidelity term. In contrast, SO and CORe require adjusting another parameter, λ₂, which controls the extent of outlier rejection. A smaller value of λ₂ corresponds to more aggressive outlier rejection at the potential cost of discarding valid data.

### Example Methods

For evaluation of the proposed reconstruction method, the study included two simulation studies with retrospective undersampling and three in vivo studies with prospective undersampling. The first simulation study was performed on a static 2D phantom. For the second simulation, the study used a digital dynamic phantom. Both simulation studies compared the four reconstruction methods: CS (Equation (2)), RR (Equation (3)), SO (Equation (5)), and CORe (Equation (6)) as discussed herein, establishing the advantage of CORe over CS, RR, and SO in the simulation studies, the study compared CORe with the conventional CS reconstruction framework (Equation (2)) in volumetric CMR reconstruction. The example studies chose [symbol] (x)=λ₁∥Ψx∥₁, with Ψ representing the undecimated wavelet transform in the spatio-temporal domain. [29] In vivo studies were performed on consented healthy subjects and patients, approved by the institutional review board (IRB).

### Example Study I: I-Static Phantom Study

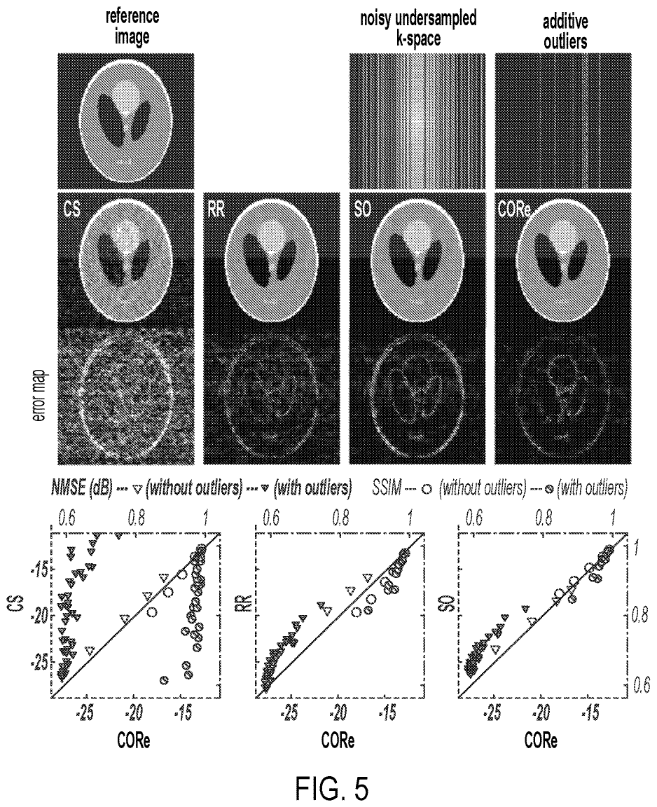

This study compared CS, RR, SO, and CORe for the reconstruction of a 128×128 Shepp-Logan phantom. To simulate single-coil k-space data, a 2D discrete Fourier transform of the digital phantom was performed, followed by Cartesian undersampling, using the golden ratio offset (GRO) sampling pattern. [30]. The net acceleration rate was fixed at 2.2. To simulate noisy measurements, circularly symmetric white Gaussian noise with a fixed variance σ² was added to the undersampled k space data, as shown in FIG. 5. To introduce outliers, a certain fraction of readouts was contaminated with additional noise. The severity of these outliers varied across 50 realizations. Specifically, the fraction of contaminated readouts was randomly chosen from a range of 1% to 20%, and the variance of the noise added to these readouts was randomly selected from a range between σ² and 10σ². Also, to assess the methods in the absence of outliers, the study performed an additional 5 realizations without outliers, each with a different noise variance ranging from σ² to 4σ². The four methods were compared in terms of normalized mean square error (NMSE) and structural similarity index (SSIM).

### Example Study II: Dynamic Phantom Study

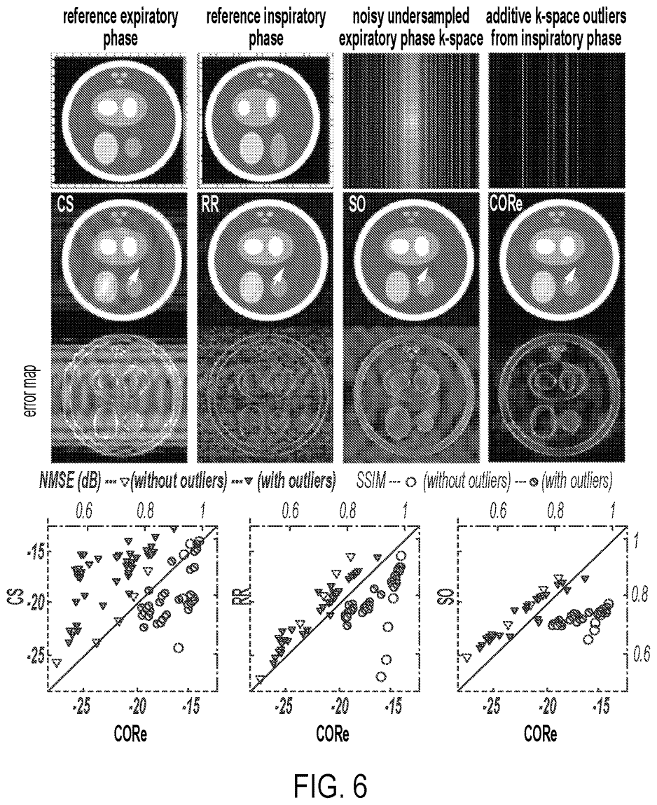

While Study I was applicable to outliers that originated from excessive additive noise, Study II was more focused on motion artifacts. This study simulated a 356×356 dynamic phantom that cycles through “inspiratory” and “expiratory” states. For simplicity, cardiac motion was not included and only two respiratory motion states and single-coil data were simulated, as shown in FIG. 6. Over a period of five respiratory cycles, a total of 360 readouts were simulated using a GRO sampling pattern similar to that employed in Study i. This resulted in a total of 130 readouts from each of the two motion states. To simulate outliers originating from imperfect data binning, the study created a contaminated undersampled k-space where 90% of the sampled readouts were derived from the expiratory state and 10% originated from the inspiratory state. The resulting motion-contaminated undersampled k-space, with added circularly symmetric white Gaussian noise of fixed variance σ², was then used to compare CS, RR, SO, and CORe reconstructions using NMSE and SSIM. The simulation was repeated 50 times, each with a random realization of the outlier locations. Similar to Study I, the study considered an additional 5 realizations for a range of noise variance, without motion contamination.

### Example Study III: High Resolution 3D Cine Study

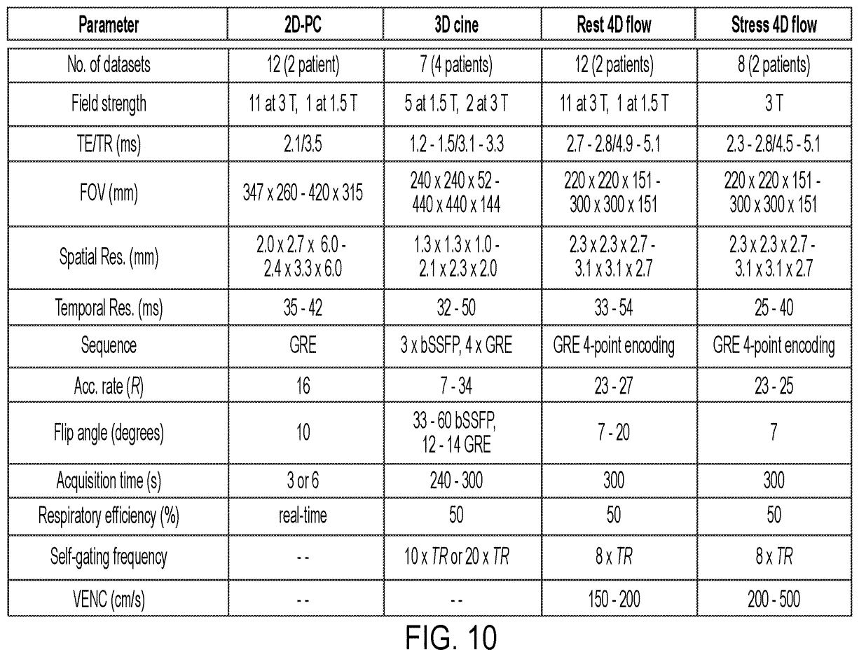

This study compared CS and CORe using seven high spatial resolution 3D cine datasets collected using a self-gated, free-running sequence with a fixed acquisition time of 5 minutes using Cartesian sampling. [30]. The field-of-view (FOV) was selected to visualize the aortic valve or to cover the whole heart. The data were collected on a clinical 1.5 T scanner (MAGNETOM Sola, Siemens Healthcare, Erlangen, Germany) and a clinical 3 T scanner (MAGNETOM Vida, Siemens Healthcare, Erlangen, Germany) equipped with 28-channel and 48 channel receive coils, respectively. Seven datasets were collected from consented subjects (age range, 23-75 years; 1 female; 4 patients, datasets #1-#4). Dataset #7 was acquired under exercise stress using a supine cycle ergometer (MR Ergometer Pedal, Lode B.V., Groningen, The Netherlands). Additional scan parameters are summarized in FIG. 10. The range of acceleration rates is large due to significant differences in FOVs and spatial resolutions across the seven datasets.

Cardiac and respiratory gatings were performed using the self-gating readouts that were repeatedly acquired in the superior-inferior (SI) direction. Self-gating lines were reorganized to form a Casorati matrix. Applying two band-pass filters along the temporal dimension of the matrix, tailored to the frequency ranges of cardiac and respiratory motion, preceded the final step of PCA to extract the motion surrogate signals. From the cardiac signal, the k-space data were binned into 20 cardiac phases for all subjects. From the respiratory signal, the k-space data belonging to the expiratory phase was selected using soft gating with an efficiency of 50%. [31] The data from the expiratory bin were used to reconstruct 3D cine images. In order to assess the quality of reconstructed images and evaluate the effectiveness of CORe in reducing motion artifacts, the study asked three expert readers to blindly score CS and CORe reconstructions. To facilitate scoring, a 2D cine series was selected from each 3D reconstruction. Each image series was scored on a five-point Likert scale (5: excellent, 4: good, 3: adequate, 2: poor, 1: nondiagnostic) for both level of artifacts and image sharpness.

### Example Study IV: Rest 4D Flow

Study IV compared CS and CORe for freerunning 4D flow imaging performed at rest. Twelve datasets (age range, 22-68 years; 4 females; 2 patients, dataset #11 and dataset #12) were collected using a self-gated, free-running research sequence with a fixed scan time of 5 minutes using a Cartesian sampling proposed by Pruitt et al.³¹ The first eleven datasets in FIG. 13 4 were acquired on a clinical 3 T scanner (MAGNETOM Vida, Siemens Healthcare, Erlangen, Germany) equipped with 48-channel receive coils. The last dataset (#12) in FIG. 13 was collected on a clinical 1.5 T scanner (MAGNETOM Sola, Siemens Healthcare, Erlangen, Germany) equipped with 28-channel receive coils. To compare net flow (ml/beat) and peak velocity (cm/s) at ascending aorta (Aao) using CORe and CS, real-time 2D phase-contrast MRI (2D-PC) was used as a reference. The imaging volume was selected to cover the whole heart and aortic arch. FIG. 10 provides a summary of the additional acquisition parameters. The cardiac binning and soft respiratory gating were performed as described in Study III. Using CORe and CS, magnitude and three velocity components were reconstructed: vₓ in the superior-inferior direction, v_{y} in the right-left direction, and v_{z} in the anterior-posterior direction. After reconstruction, 4D flow images underwent background phase correction.³² Then, the images were converted to DICOM format and analyzed in CAAS (Pie Medical Imaging B.V., Maastricht, The Netherlands) for flow assessment.

### Example Study V: V-Stress 4D Flow

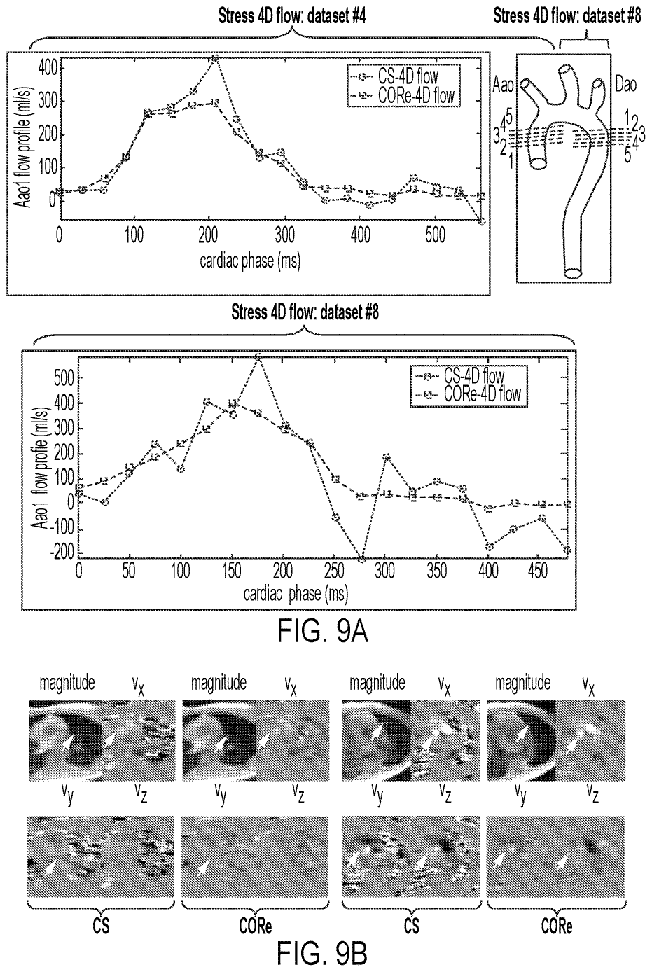

Study V, performed 4D flow imaging to evaluate the efficacy of CORe in comparison to CS for mitigating motion artifacts in the presence of exercise-induced motion. Eight datasets (age range, 26-68 years; 2 females; 2 patients, datasets #7 and #8) were acquired during in-magnet exercise using the research sequence as described in Study IV. All datasets were collected on a clinical 3 T scanner (MAGNETOM Vida, Siemens Healthcare, Erlangen, Germany) equipped with a supine cycle ergometer (MR Ergometer Pedal, Lode B.V., Groningen, The Netherlands). FIG. 10 provides a summary of the additional acquisition parameters. The DICOM images were reconstructed using CS and CORe, as described previously in Study IV. In the absence of a reference 2D-PC under exercise stress, the study compared CS and CORe by evaluating the mean and standard deviation (SD) of net flow across five transecting planes perpendicular to the flow direction at both the ascending and descending aorta, as depicted in FIG. 9A. These parallel planes were positioned closely, yet away from any vessel bifurcation, maintaining an approximate distance of 6-8 mm between adjacent planes. Therefore, in the absence of motion artifacts, one would expect the net flow through the five slices to be consistent.

### Image Reconstruction

All reconstruction methods were implemented using the ADMM algorithm. [33, 34]. The derivation of the ADMM algorithm to solve Equation (6) is given in Section (B) in Appendix. In each outer iteration of ADMM, a sequence of simpler subproblems is solved either in closed form or using a small number of gradient descent iterations. Study I utilized 500 outer iterations, and the image reconstruction time with CPU processing was approximately 9 seconds for each method. Study II used 350 outer iterations, and the image reconstruction time with CPU processing was approximately 30 seconds for each method. For all in vivo studies, coil compression was performed to generate 12 virtual coils for faster processing, [35] and the coil sensitivity maps were estimated using the method by Walsh et al. [36] Image reconstruction was performed using 50 outer iterations on an NVIDIA RTX 3090 GPU. The reconstruction times for both CS and CORe in Studies III, IV and V were 25, 11 and 11 minutes each, respectively. All images were reconstructed offline using MATLAB (Mathworks, Natick, Massachusetts).

In Study I and Study II, the hyperparameters, i.e., λ₁ for CS and RR and λ₁ and λ₂ for SO and CORe, were optimized using two additional realizations, one with and one without the outliers. The optimization aimed to minimize the overall NSME (in dB) defined as

$$20 {log}_{10} ( \frac{{‖ \hat{x} - x ‖}_{2}}{{‖ x ‖}_{2}} ) .$$

For the 3D cine study, the hyperparameters for CS and CORe were selected based on visual inspection of a separate dataset, which was not included in the current study. For 4D flow analysis, the hyperparameters were chosen to minimize the bias from 2D-PC net flow value using another dataset not included in this study.

### Results

### Example Study I: Static Phantom

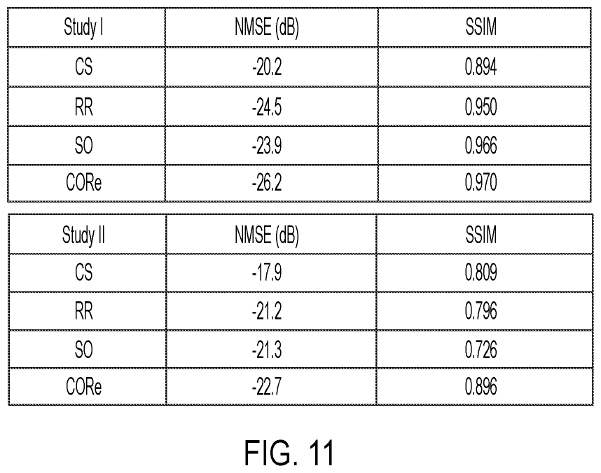

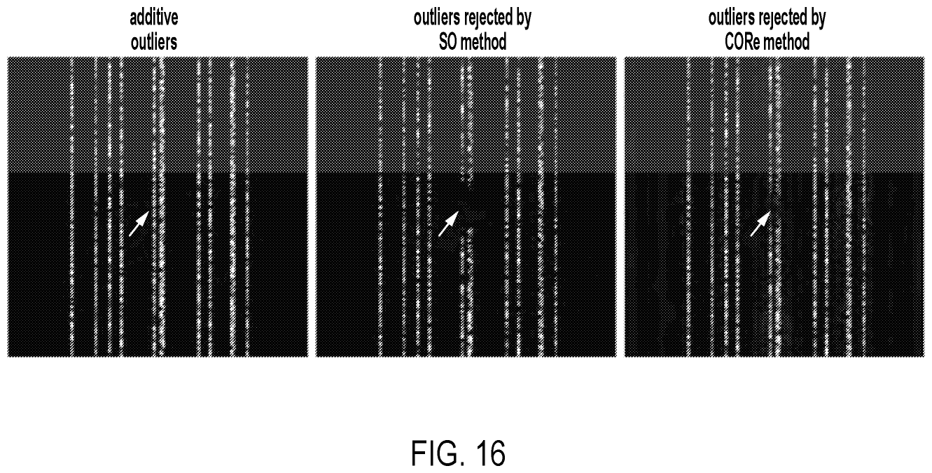

FIG. 5 summarizes the results of Study I. The top row shows the noiseless reference image, noisy undersampled k-space, and additive outliers for one representative realization. The second and third rows show reconstructions from CS, RR, SO, and CORe and their respective error maps after three-fold amplification. The bottom row shows scatter plots for 55 realizations, comparing CORe with CS, RR, and SO in terms of NMSE and SSIM. The averaged NMSE and SSIM values are illustrated in FIG. 11, with bold values representing the best results. Statistical analysis using paired sample t-test with α=0.01 and Bonferroni correction reveals that CORe exhibits significant improvement over CS, RR, and SO in terms of both NMSE (dB) and SSIM. FIG. 16 visually compares outliers rejected by SO and CORe for a representative case in this study. Although this simulation study is not directly tied to motion-related outliers, it demonstrates the merit of CORe for a broader application, where some of the readouts are corrupted by higher variance noise.

### Example Study II: Dynamic Phantom

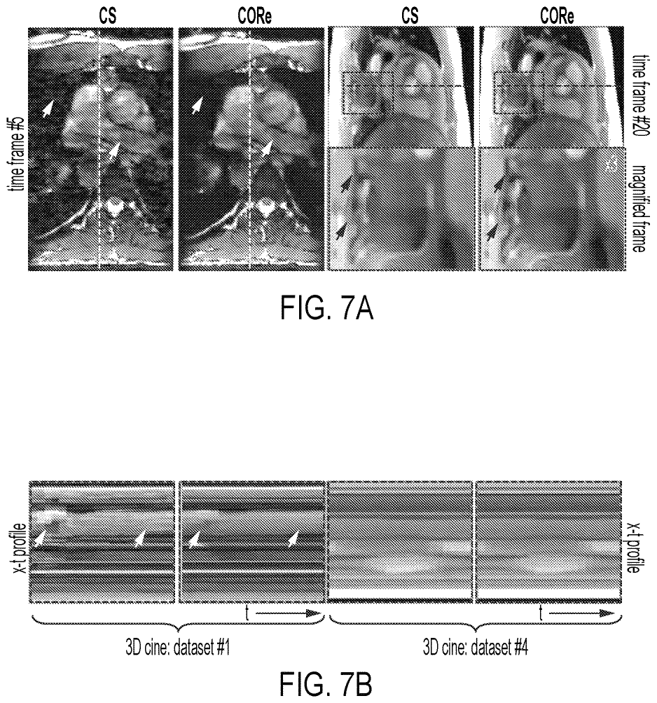

FIGS. 7A-7B summarize the results of Study II. The top row shows the reference inspiratory and expiratory motion states of the bimodal phantom, noisy undersampled kspace from the expiratory phase, and additive motion outliers from the inspiratory phase for one representative realization. The second row shows the reconstructed images from CS, RR, SO, and CORe with arrows highlighting the motion artifacts. The third row shows the respective error maps after three-fold amplification. The bottom row shows scatter plots for 55 realizations, comparing CORe with CS, RR, and SO in terms of NMSE and SSIM. The averaged NMSE and SSIM values are reported in FIG. 11, with bold values representing the best results. Statistical analysis using paired sample test with α=0.01 and Bonferroni correction underscores that CORe exhibits significant improvement over CS, RR, and SO in terms of both NMSE (dB) and SSIM.

### Example Study III: High Resolution 3D Cine

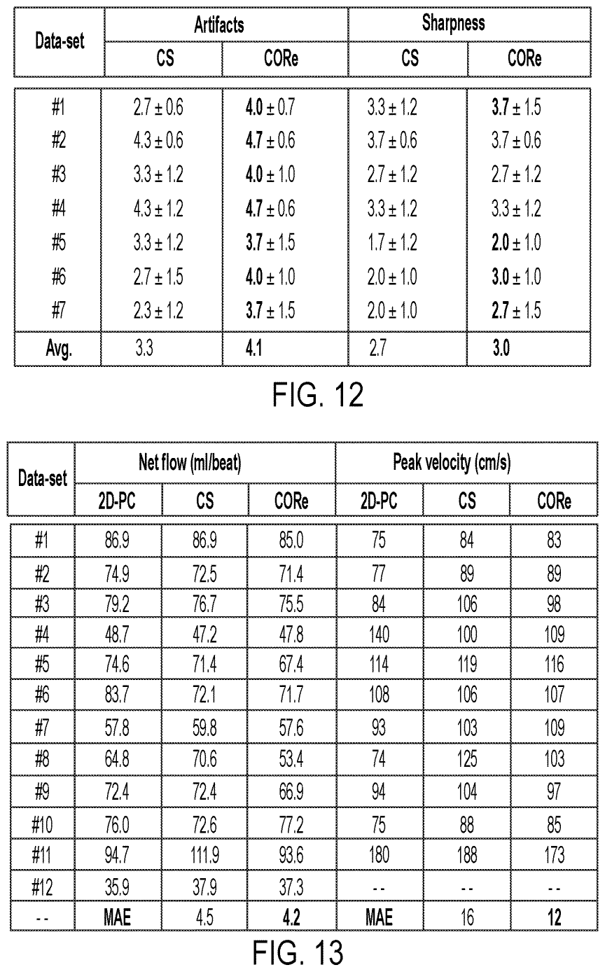

FIG. 12 presents the averaged scores obtained from the blinded reader evaluation of the reconstructed image series, with the best scores in bold font. Our analysis compared CORe and CS using a mixed-effects model with Bonferroni correction. The results indicate that CORe outperforms CS in terms of artifact reduction and image sharpness, with these differences being statistically significant for α=0.01. FIG. 7A provides a visual comparison of the 3D cine reconstructions from CS and CORe, showing a representative frame from two different datasets (#1 and #4). FIG. 7A further illustrates visual differences between CS and CORe in terms of artifacts or image sharpness. FIG. 7B compares x-t profiles for locations highlighted with green lines in FIG. 7A.

### Example Study IV: Rest 4D Flow

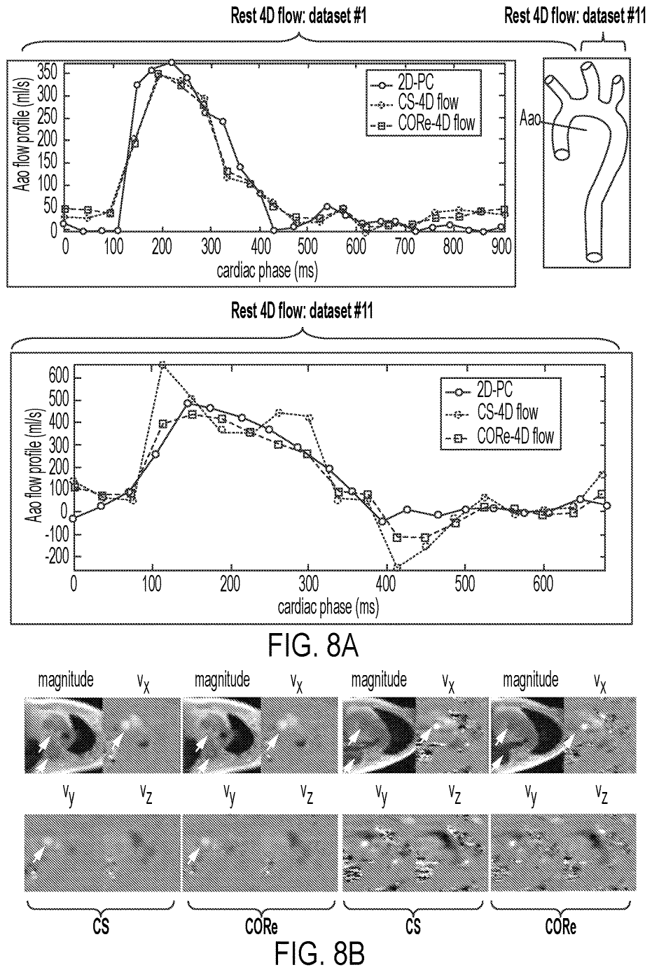

FIG. 13 compares net flow quantification (ml/beat) and peak velocities (cm/s) from CS and CORe with 2D-PC serving as reference. The flow measurements were performed at an Aao plane depicted in FIG. 8A. FIG. 13 shows the mean absolute error (MAE) of CS and CORe values from the 2D-PC reference. A paired sample t-test, with Bonferroni correction, indicates that the differences of both CS and CORe from 2D-PC are statistically insignificant at a significance level of α=0.01. FIG. 8A provides a visual comparison of two representative flow rate profiles from CS and CORe. FIG. 8B shows the magnitude and three velocity components corresponding to datasets #1 and #11. A single frame at systole (peak flow) is shown from a 2D slice transecting Aao as depicted in FIG. 8A. The arrows highlight areas where CS exhibits motion artifacts or blurring. Peak velocity assessment from dataset #12 was not performed in this example due to localized signal loss associated with a prosthetic aortic valve.

### Example Study V: Stress 4D Flow

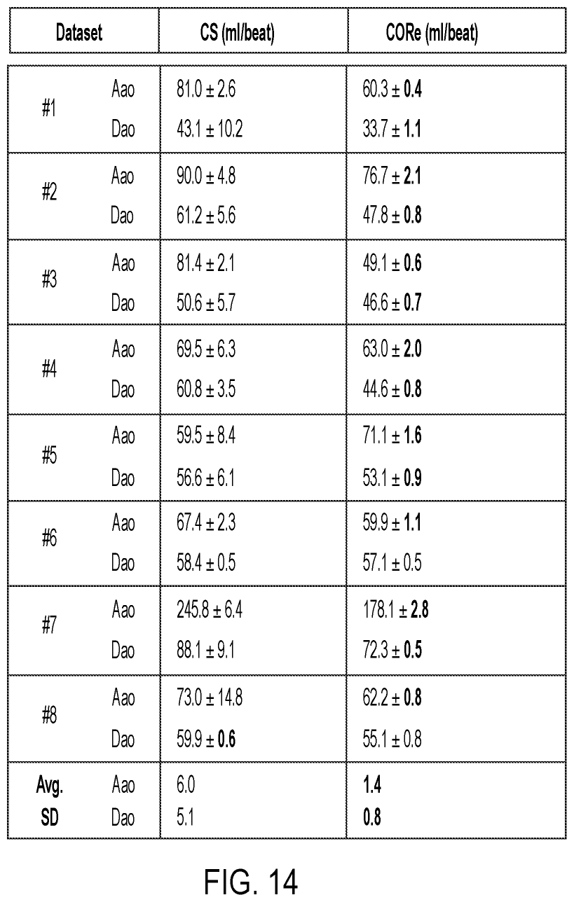

FIG. 14 compares CS and CORe for mean±SD values of net flow quantification (ml/beat) across the Aao and Dao planes depicted in FIG. 9A. The lower SD values are bolded. For α=0.01, a paired sample t-test indicates a statistically significant difference in standard deviations between CORe and CS. FIG. 9A provides a visual comparison of two representative flow rate profiles from CS and CORe at Aao plane 1. FIG. 9B shows the magnitude and three velocity components corresponding to datasets #4 and #8. A single frame at systole (peak flow) is shown from Aao plane 1. The arrows highlight areas where CS exhibits motion artifacts or blurring. Video was collected including CS and CORe reconstructed cine image series for magnitude, vₓ, v_{y}, and v_{z} components of dataset #8. The video included 20 frames from one cardiac cycle, played four times at a frame rate of 10 frames per second.

### Discussion

Free-running self-gated volumetric CMR is prone to artifacts due to the presence of outliers in binned k-space data. Embodiments of the present disclosure overcome these artifacts and improve reconstruction using improved reconstruction methods, including the example embodiment referred to herein as CORe. CORe can effectively integrate outlier rejection into compressive reconstruction (e.g., using the relationships illustrated in equation 6). CORe leverages the group sparse behavior of outliers in k-space to separate them from properly binned measurements, resulting in reduced artifacts.

The simulation studies herein quantitatively compare CORe with CS (Equation (2)), RR (Equation (3)), and SO (Equation (5)). Study I simulated noisy undersampled k-space from a static phantom and contaminate a fraction of the readouts with stronger additive complex noise to mimic outliers. The reconstruction methods were assessed with and without the presence of outliers in the data using NMSE and SSIM metrics. The results from 55 realizations demonstrate that CS reconstructions suffer significant quality degradation in the presence of outliers. While RR and SO methods exhibit noticeable improvement over CS, CORe outperforms them, achieving the best average NMSE and SSIM values, as presented in FIG. 11. Study II used undersampled k-space readouts are combined from two different motion states of a dynamic phantom to mimic free-running CMR acquisition with imperfect binning. Again, in terms of NMSE and SSIM averaged over 55 realizations, CORe outperforms CS, RR, and SO, as reported in FIG. 11. The error maps also highlight residual motion artifacts in CS, RR and SO that are not visible in CORe. FIG. 16 highlights the benefit of group sparsity used in CORe over unstructured sparsity used in SO. FIG. 16 shows the error maps, \hat{v} represented in k-space for one of the examples in Study I. Compared to CORe, SO is unable to reject the entire readouts that are outliers.

Study III evaluated CORe for reconstructing high-resolution 3D cine images. The comparison is made with CS, which is a common choice for FRV reconstruction. The study did not include RR and SO as these methods have not been previously proposed or validated for MRI reconstruction. The reconstructed images from CORe and CS were blindly scored by three expert readers on the criteria of artifacts and sharpness. The results from the reader study (presented in FIG. 12) show that CORe not only outperforms CS in terms of artifacts but also provides marginal but consistent improvement in image sharpness. The suppression of artifact by CORe is evident in FIG. 7B and also was evident in video data collected during the study. The improvement in sharpness by CORe is evident in FIG. 7B. The simultaneous reduction in artifacts and improvement in sharpness by CORe indicate that the relatively inferior performance of CS cannot be attributed to suboptimal selection of the regularization strength, λ₁. It is well known that the regularization strength provides a trade-off between artifact (or noise) suppression and image sharpness. [37] For CS, increasing λ₁ to suppress artifacts would lead to further image blurring, and decreasing λ₁ to improve image sharpness would further amplify image artifacts.

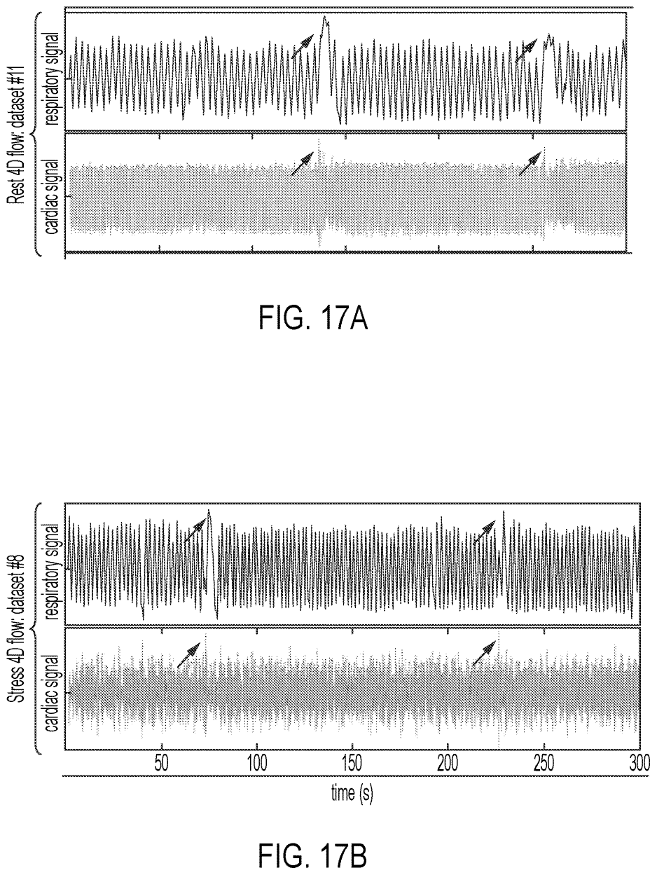

Study IV evaluates CS and CORe for the reconstruction of 4D flow data acquired at rest, using 2D-PC as a reference. In particular, the study quantified the net flow (ml/beat) and peak velocity (cm/s) across a plane transecting the ascending aorta (Aao) as demonstrated in FIG. 8A. The flow quantification results presented in FIG. 13 demonstrates that both CS and CORe reconstructions exhibit agreement with 2D-PC measurements. The MAE values indicate that CS and CORe produce comparable results, with CORe showing a marginal reduction in bias. However, in a few datasets, CORe underestimates the net flow and CS tends to overestimate in comparison to the reference. For (patient) dataset #11, CS demonstrates a significant overestimation of net flow when compared to the reference, evident from the oscillations observed in the CS reconstructed flow rate profile (ml/s) depicted in FIG. 8A. This jagged flow behavior can be attributed to the potential incorrect binning of k space data, likely caused by bulk motion by the subject during the scan. This interpretation is supported by the cardiac and respiratory signals displayed in FIG. 17A. On the contrary, the flow profile from CORe reconstruction for the same dataset is consistent with the 2D-PC flow profile, demonstrating CORe's ability to mitigate motion-induced outliers in k-space data. Additionally, the visual comparison of magnitude and velocity components in FIG. 8B shows that CORe reconstruction effectively reduces motion artifacts while preserving finer details compared to CS.

Study V assesses CS and CORe for the reconstruction of 4D flow data acquired during exercise stress. Since the self-gating signal can be less reliable under exercise stress, the k-space data collected under exercise stress are more susceptible to motion outliers. The study compared the consistency of flow across multiple planes transecting Aao and Dao regions as depicted in IG. 9A. The flow quantification results presented in FIG. 14 shows that CORe demonstrates considerably lower variation in net flow values across the planes compared to CS. The representative flow profiles shown in FIG. 9A illustrates that flow profiles obtained from CORe images appear smoother and more physiologically realistic. In contrast, CS flow profiles exhibit more jagged behavior, potentially due to uncompensated motion outliers. This interpretation is supported by the cardiac and respiratory signals shown for dataset #8 in FIG. 17B. The improvement offered by CORe in mitigating motion artifacts can be clearly observed in the representative 4D flow images presented in FIG. 9B. This demonstrates that the impact of CORe becomes even more pronounced in stress 4D flow imaging.

Although the examples herein use CORe applied to Cartesian sampling, it should be understood that CORe and other implementations of the present disclosure are equally applicable to non-Cartesian sampling and/or applications other than cine and flow. Also, CORe optionally makes no assumption about the genesis of outlier readouts and can potentially suppress outliers originating from sources other than motion, such as RF interference and flow artifacts. Finally, the implementations of the present disclosure, including CORe, can be integrated with deep-learning-based reconstruction methods [38] and motion correction-based reconstruction methods [13] by modifying the data-consistency term.

CORe implementation using the ADMM algorithm does not significantly increase computational cost over CS. The reconstruction times for CORe and CS are comparable. Still, this work has several limitations. First, compared to CS, CORe introduces another tuning parameter, λ₂. In all our studies, this parameter was optimized using an additional dataset that was not included in the comparison. For applications, where imaging parameters vary widely across subjects, using one value of λ₂ may not be optimal. Employing a smaller value for λ₂ than the optimal choice may risk discarding valid k-space measurements. Conversely, selecting a larger value for λ₂ may diminish the advantage of CORe over CS in terms of outlier suppression. Second, CORe relies on the assumption that coil sensitivity maps, which are estimated from the measured k-space that includes outliers, are of high quality. In cases where this assumption breaks down, the final quality of CORe reconstruction may not be adequate. A possible solution to this problem is to estimate the coil sensitivity maps again after a preliminary CORe reconstruction and then perform a final CORe reconstruction using the updated maps. This remedy, however, would come at the cost of increased reconstruction time. Third, the in vivo studies include a limited number of subjects, with evaluation relying on subjective assessment of image quality in Study III, quantification of only two hemodynamic parameters in Study IV, and internal consistency in Study V. Since artifacts can significantly impact quantification of advanced hemodynamic parameters, including wall shear stress and pressure gradients, CORe is expected to improve the reliability of such parameters. Future studies will include larger in vivo studies, quantitative comparison of cardiac function to 2D imaging, and assessment of advanced hemodynamic parameters.

### Example 3

Example methods were developed to implement CORe (e.g., example 2) and other implementations of the present disclosure.

Consider model in Equation (4), i.e.,

$$y = Ax + v + ω \qquad ( A1 )$$

Assuming that x, v, and ω are realizations of random variables X, V, and Ω defined by probability density functions p(x)∝exp (− [symbol] (x)), p(v)∝exp (−λ₂∥v∥₁), and p(ω)∝exp (−∥w∥/σ²), respectively. With these assumptions, it is easy to see that y is a realization of a random variable Y with p(y)∝exp (−∥y−(Ax+v)∥/σ²). By Bayes' rule, the posterior probability p(x, v|y) is given by

$$p ( x , v \vert y ) = \frac{p ( y \vert x , v ) p ( x , v )}{p ( y )} \qquad ( A2 )$$

By ignoring the denominator in Equation (A2) as it does not depend on x or v and assuming X and V are independent,

p(x, v|y) ∝p(y|x, v)p(x)p(v)(A3)

By taking the negative log of Equation (A3) and minimizing it with respect to x and v,

$${\hat{x}}_{SO} = {arg min}_{x , v} \frac{1}{{σ}^{2}} {‖ Ax - y + v ‖}_{2}^{2} + ℛ ( x ) + {λ}_{2} {‖ v ‖}_{1} \qquad \# ( A4 )$$

which is the same as Equation (5). It also easy to see that by replacing p(v)∝exp (−λ₂∥v∥₁) with p(v)∝exp (−λ₂∥g(v)∥₁), arrives at the CORe formulation in Equation (6). Therefore, \hat{x}_{SO} and \hat{x}_{CO} can be viewed as MAP estimates under (i) circularly symmetric white Gaussian noise, (ii) sparse or group sparse prior on V, (iii) prior [symbol] (x) on X, and (iv) statistical independence between X and V.

### Example ADMM Algorithm

Reconsider the optimization problem in Equation (6) with [symbol] (x)=λ₁∥Ψx∥₁, i.e.,

$${min}_{x , v} \frac{1}{{σ}^{2}} {‖ Ax - y + v ‖}_{2}^{2} + {λ}_{1} {‖ Ψ x ‖}_{1} + {λ}_{2} {‖ g ( v ) ‖}_{1} \qquad ( B5 )$$

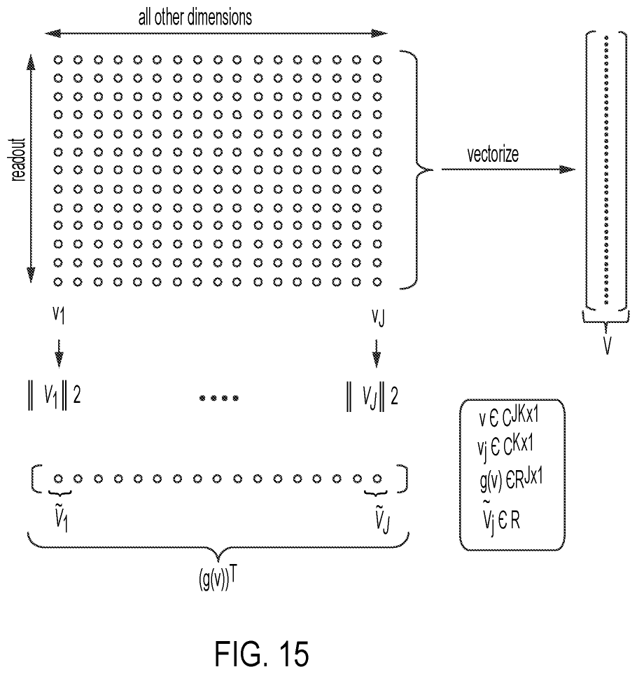

represents samples in the j^{th} readout as shown in FIG. 15.
By w₁≙Ψx and w₂≙g(v), the problem in Equation (B5) can be equivalently written as

$${min}_{x , v} \frac{1}{{σ}^{2}} {‖ Ax - y + v ‖}_{2}^{2} + {λ}_{1} {‖ {w}_{1} ‖}_{1} + {λ}_{2} {‖ {w}_{2} ‖}_{1} subject to Ψ x = {w}_{1} , \\ g ( v ) = {w}_{2} \qquad ( B6 )$$

The augmented Lagrangian for this problem can be expressed as

$${ℒ}_{{μ}_{1} , {μ}_{2}} ( x , v , {w}_{1} , {w}_{2} , {ρ}_{1} , {ρ}_{2} ) = \frac{1}{{σ}^{2}} {‖ Ax - y + v ‖}_{2}^{2} + {λ}_{1} {‖ {w}_{1} ‖}_{1} + {λ}_{2} {‖ {w}_{2} ‖}_{1} + Re \{ {ρ}_{1}^{H} ( Ψ x - {w}_{1} ) \} + \frac{{μ}_{1}}{2} {‖ Ψ x - {w}_{1} ‖}_{2}^{2}$$

By u₁≙ρ₁/μ₁ and u₂≙ρ₂/μ₂, the expression in Equation (B7) can simplify to

$${ℒ}_{{μ}_{1} , {μ}_{2}} ( x , v , {w}_{1} , {w}_{2} , {u}_{1} , {u}_{2} ) = \frac{1}{{σ}^{2}} {‖ Ax - y + v ‖}_{2}^{2} + {λ}_{1} {‖ {w}_{1} ‖}_{1} + {λ}_{2} {‖ {w}_{2} ‖}_{1} + \frac{{μ}_{1}}{2} {‖ Ψ x - {w}_{1} + {u}_{1} ‖}_{2}^{2} + \frac{{μ}_{1}}{2} {‖ {u}_{1} ‖}_{2}^{2}$$

Now, the primal problem in Equation (B6) can be solved by this minimax problem

$${min}_{x , v , {w}_{1} , {w}_{2}} {max}_{{u}_{1} , {u}_{2}} \{ {ℒ}_{{μ}_{1} , {μ}_{2}} ( x , v , {w}_{1} , {w}_{2} , {u}_{1} , {u}_{2} ) \} \qquad ( B9 )$$

which leads to core that tackles the problem presented in Equation (B9) by breaking it down into simpler subproblems that update one variable at a time.

The subproblem in update-x of core consists of two quadratic terms and thus has a closed-form solution. For larger problems where explicit matrix representations of A and Ψ are not feasible, a practical alternative is to solve this problem using gradient descent iterations, with the gradient direction specified by

$$\frac{2}{{σ}^{2}} {A}^{H} ( Ax - y + {v}^{( t - 1 )} ) + {μ}_{1} {Ψ}^{H} ( Ψ x - {w}_{1}^{( t - 1 )} + {u}_{1}^{( t - 1 )} ) .$$

The subproblem in update-w1 of core admits a closed-form solution given by

$${w}_{1}^{( t )} = {𝒮}_{{λ}_{1} / {μ}_{1}} ( Ψ {x}^{( t )} + {u}_{1}^{( t - 1 )} ) , where {𝒮}_{{λ}_{1} / {μ}_{1}} ( z ) = \frac{z}{\vert z \vert} max ( \vert z \vert - {λ}_{1} / {μ}_{1} , 0 )$$

is the element-wise soft-thresholding, with |z|representing the element-wise absolute values. Likewise, the subproblem in update-w2 of core also admits a closed-form solution given by

$${w}_{2}^{( t )} = {𝒮}_{{λ}_{2} / {μ}_{2}} ( g ( {v}^{( t )} ) + {u}_{2}^{( t - 1 )} ) .$$

The gradient direction for the second term in update-v of core may not be obvious. Here, the gradient direction for this subproblem is outlined. By

$${r}_{2}^{( t - 1 )} {=}^{Δ} {w}_{2}^{( t - 1 )} - {u}_{2}^{( t - 1 )} ,$$

the second term of this subproblem can be presented by:

$$\begin{matrix}H {=}^{Δ} \frac{{μ}_{2}}{2} {‖ g ( v ) - {r}_{2}^{( t - 1 )} ‖}_{2}^{2} \\ = \frac{{μ}_{2}}{2} {\sum }_{j = 1}^{J} {( {\tilde{v}}_{j} - {r}_{2 , j}^{( t - 1 )} )}^{*} ( {\tilde{v}}_{j} - {r}_{2 , j}^{( t - 1 )} )\end{matrix}$$

where

$${r}_{2 , j}^{( t - 1 )}$$

is the j^{th} element of

$${r}_{2}^{( t - 1 )}$$

and (⋅)* represents complex conjugate. The gradient of H using Wirtinger derivatives with respect to

$${v}_{l}^{*} , {}^{39} where {v}_{l}^{*}$$

is element-wise complex conjugate of vₗ, is given by:

$$\begin{matrix}{∇}_{l} H = 2 \frac{\partial H}{\partial {v}_{l}^{*}} = 2 \frac{\partial }{\partial {v}_{l}^{*}} ( \frac{{μ}_{2}}{2} {\sum }_{j = 1}^{J} {( {‖ {v}_{j} ‖}_{2} - {r}_{2 , j}^{( t - 1 )} )}^{*} ( {‖ {v}_{j} ‖}_{2} - {r}_{2 , j}^{( t - 1 )} ) ) \\ = {μ}_{2} \frac{\partial }{\partial {v}_{l}^{*}} ( {( {‖ {v}_{l} ‖}_{2} - {r}_{2 , l}^{( t - 1 )} )}^{*} ( {‖ {v}_{l} ‖}_{2} - {r}_{2 , l}^{( t - 1 )} ) ) \\ = {μ}_{2} ( {v}_{l} - \frac{1}{2} ( {r}_{2 , l}^{* ( t - 1 )} + {r}_{2 , l}^{( t - 1 )} ) \frac{{v}_{l}}{{‖ {v}_{l} ‖}_{2}} )\end{matrix}$$

with the gradient across all readouts given by ∇H=[(∇₁H)^{T}, (∇₂H)^{T}, . . . , (∇ⱼH)^{T}]^{T}∈ [symbol] ^{JK×1}. By adding ∇H to the gradient from the first term in update-v of core, the gradient direction for this subproblem is obtained as

$$\frac{2}{{σ}^{2}} ( {Ax}^{( t )} - y + v ) + ∇ H .$$

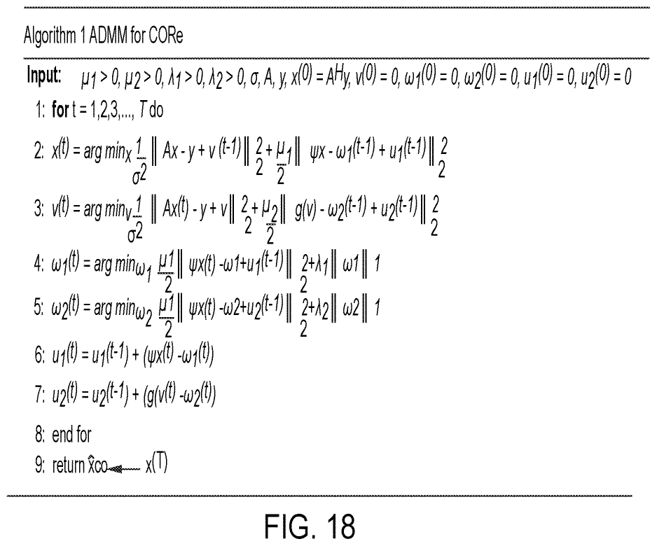

FIG. 18 illustrates an example alternating direction method of multipliers optimization method (ADMM), according to implementations of the present disclosure.

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

### REFERENCES

[1] Leiner T, Bogaert J, Friedrich M G, Mohiaddin R, Muthurangu V, Myerson S, et al. SCMR Position Paper (2020) on clinical indications for cardiovascular magnetic resonance. Journal of Cardiovascular Magnetic Resonance. 2020; 22:1-37.
[2] Paling M R, Brookeman J R. Respiration artifacts in MR imaging: reduction by breath holding. Journal of Computer Assisted Tomography. 1986; 10(6):1080-1082.
[3] Atkinson D J, Edelman R. Cineangiography of the heart in a single breath hold with a segmented turboFLASH sequence. Radiology. 1991; 178(2):357-60.
[4] Stuber M, Scheidegger M, Boesiger P. Realtime imaging of the heart. In Proceedings of the 5th Annual Meeting of ISMRM, Vancouver, BC, Canada. 1997.
[5] Kerr A B, Pauly J M, Nishimura D G. Partial k-space acquisition for real-time MR imaging. In Proceedings of the 6th Annual Meeting of ISMRM, Sydney, Australia. 1998.
[6] Ehman R L, McNamara M, Pallack M, Hricak H, Higgins C. Magnetic resonance imaging with respiratory gating: techniques and advantages. AJR American journal of roentgenology. 1984; 143(6):1175-82.
[7] Uribe S, Muthurangu V, Boubertakh R, Schaeffter T, Razavi R, Hill D, et al. Whole-heart cine MRI using real-time respiratory self-gating. Magnetic Resonance in Medicine. 2007; 57(3):606-13.
[8] Di Sopra L, Piccini D, Coppo S, Stuber M, Yerly J. An automated approach to fully self-gated free-running cardiac and respiratory motion-resolved 5D whole-heart MRI. Magnetic Resoance in Medicine. 2019; 82(6):211832.
[9] Larson A C, Kellman P, Arai A, Hirsch G A, McVeigh E, Li D, et al. Preliminary investigation of respiratory selfgating for free-breathing segmented cine MRI. Magnetic Resonance in Medicine. 2005; 53(1):159-68.
[10] Feng L, Axel L, Chandarana H, Block K T, Sodickson D K, Otazo R. XD-GRASP: Golden-angle radial MRI with reconstruction of extra motion-state dimensions using compressed sensing. Magnetic Resonance in Medicine. 2016; 75(2):775-88.
[11] Feng L, Coppo S, Piccini D, Yerly J, Lim R P, Masci P G, et al. 5D whole-heart sparse MRI. Magnetic Resonance in Medicine. 2018; 79(2):826-38.
[12] Bhat H, Ge L, Nielles-Vallespin S, Zuehlsdorff S, Li D. 3D radial sampling and 3D affine transform-based respiratory motion correction technique for free-breathing whole-heart coronary MRA with 100% imaging efficiency. Magnetic Resonance in Medicine. 2011; 65(5):1269-77.
[13] Ingle R R, Wu H H, Addy N O, Cheng J Y, Yang P C, Hu B S, et al. Nonrigid autofocus motion correction for coronary MR angiography with a 3D cones trajectory. Magnetic Resonance in Medicine. 2014; 72(2):347-61.
[14] Ludwig J, Speier P, Seifert F, Schaeffter T, Kolbitsch C. Pilot tone-based motion correction for prospective respiratory compensated cardiac cine MRI. Magnetic Resonance in Medicine. 2021; 85(5):2403-16.
[15] Chen C, Liu Y, Simonetti O P, Tong M, Jin N, Bacher M, et al. Cardiac and respiratory motion extraction for MRI using pilot tone-a patient study. The International Journal of Cardiovascular Imaging. 2023:1-13.
[16] Gerche A L, Claessen G, Van de Bruaene A, Pattyn N, Van Cleemput J, Gewillig M, et al. Cardiac MRI: a new gold standard for ventricular volume quantification during high-intensity exercise. Circulation: Cardiovascular Imaging. 2013; 6(2):329-38.
[17] Lustig M, Donoho D L, Santos J M, Pauly J M. Compressed Sensing MRI. IEEE Signal Processing Magazine. 2008; 25(2):72-82.
[18] Dong B, Ji H, Li J, Shen Z, Xu Y. Wavelet frame based blind image inpainting. Applied and Computational Harmonic Analysis. 2012; 32(2):368-79.
[19] Lustig M, Donoho D, PaulyJM. Sparse MRI: The application of compressed sensing for rapid MR imaging. Magnetic Resonance in Medicine. 2007; 58(6):1182-95.
[20] Lustig M, Santos J M, Donoho D L, Pauly J M. k-t Sparse: High frame rate dynamic MRI exploiting spatio-temporal sparsity. In Proceedings of the 14th Annual Meeting of ISMRM, Seattle, WA, USA. 2006:2420.
[21] Rudin L I, Osher S, Fatemi E. Nonlinear total variation based noise removal algorithms. Physica D. 1992; 60(5):359-68.
[22] Jaspan O N, Fleysher R, Lipton M L. Compressed sensing MRI: a review of the clinical literature. The British Journal of Radiology. 2015; 88(1056):20150487.
[23] Vishnevskiy V, Walheim J, Kozerke S. Deep variational network for rapid 4D flow MRI reconstruction. Nature Machine Intelligence. 2020; 2(5):328-35.
[24] Ravishankar S, Ye J C, Fessler J A. Image reconstruction: From sparsity to data-adaptive methods and machine learning. Proceedings of the IEEE. 2019; 108(1):86-109.
[25] Bustin A, Fuin N, Botnar R M, Prieto C. From compressed-sensing to artificial intelligence-based cardiac MRI reconstruction. Frontiers in Cardiovascular Medicine. 2020; 7:17.
[26] Nikolova M. A Variational Approach to Remove Outliers and Impulse Noise. Journal of Mathematical Imaging and Vision. 2004; 20(1):99-120.
[27] Bach F. Consistency of the Group Lasso and Multiple Kernel Learning. Journal of Machine Learning Research. 2008; 9:1179-325.
[28] Huang J, Zhang T. The benefit of group sparsity. The Annals of Statistics. 2010; 38(4):1978 2004.
[29] Kayvanrad M H, McLeod A J, Baxter J S H, McKenzie C A, Peters T M. Stationary wavelet transform for under-sampled MRI reconstruction. Magnetic Resonance Imaging. 2014; 32(10):1353-64.
[30] Joshi M, Pruitt A, Chen C, Liu Y, Ahmad R. Technical Report (v1.0)-Pseudo-random Cartesian Sampling for Dynamic MRI. arXiv preprint arXiv:220603630. 2022.
[31] Pruitt A, Rich A, Liu Y, Jin N, Potter L, Tong M, et al. Fully self-gated whole-heart 4D flow imaging from a 5-minute scan. Magnetic Resonance in Medicine. 2021; 85(3):1222-36.
[32] Pruitt A A, Jin N, Liu Y, Simonetti O P, Ahmad R. A method to correct background phase offset for phasecontrast MRI in the presence of steady flow and spatial wrap-around artifact. Magnetic resonance in medicine. 2019; 81(4):2424-38.
[33] Glowinski R, Marroco A. Sur l'approximation, par éléments finis d'ordre un, et la résolution, par pénalisation-dualité d'une classe de problèmes de Dirichlet non linéaires. Revue française d'automatique, informatique, recherche opérationnelle Analyse numérique. 1975; 9(R2):41-76.
[34] Goldstein T, O'Donoghue B, Setzer S, Baraniuk R. Fast Alternating Direction Optimization Methods. SIAM Journal on Imaging Sciences. 2014; 7(3):1588-623.
[35] Buehrer M, Pruessmann K P, Boesiger P, Kozerke S. Array compression for MRI with large coil arrays. Magnetic Resonance in Medicine. 2007; 57(6):1131-9.
[36] Walsh D O, Gmitro A F, Marcellin M W. Adaptive reconstruction of phased array MR imagery. Magnetic Resonance in Medicine. 2000 May; 43(5):682-90.
[37] Khare K, Hardy C J, King K F, Turski P A, Marinelli L. Accelerated MR imaging using compressive sensing with no free parameters. Magnetic Resonance in Medicine. 2012; 68(5):1450-7.
[38] Aggarwal H K, Mani M P, Jacob M. MoDL: Model-based deep learning architecture for inverse problems. IEEE Transactions on Medical Imaging. 2018; 38(2):394-405.
[39] Kreutz-Delgado K. The complex gradient operator and the CR-calculus. arXiv preprint arXiv:09064835. 2009.

What is claimed:

1. A computer-implemented method for image reconstruction, the method comprising:
receiving k-space data from an magnetic resonance imaging (MRI) machine;
sorting the k-space data into a plurality of bins, each of the plurality of bins corresponding to a respective phase of a respiratory cycle;
identifying a plurality of outliers in the k-space data; and
reconstructing an image from the k-space data using a compressive sensing (CS) technique configured to remove the plurality of outliers from the k-space data, wherein the CS technique is configured to apply structured sparsity regularization to the plurality of outliers in the k-space data.

2. The computer-implemented method of claim 1, wherein the CS technique comprises a data fidelity term and a regularization term, wherein the regularization term applies the structured sparsity regularization to the plurality of outliers in the k-space data.

3. The computer-implemented method of claim 1, wherein the CS technique is performed according to
$$\hat{x} = arg {min}_{x , v} \{ \frac{1}{2} {‖ Ax - ( y - v ) ‖}_{2}^{2} + {λ}_{1} {‖ Wx ‖}_{1} + {λ}_{2} {‖ v ‖}_{2 , 1} \} , \\ where \hat{x} = Reconstructed Image y = Collected K Space Data A = Forward Operator W = Sparsifying ( NDW ) Transform v = Rejected Outlier data points and {λ}_{i} = Tuning Parameters .$$

4. The computer-implemented method of claim 1, wherein identifying the plurality of outliers in the k-space data comprises identifying one or more outliers in the k-space data in each of the plurality of bins.

5. The computer-implemented method of claim 1, wherein the reconstructed image is a motion compensated image.

6. The computer-implemented method of claim 1, wherein the k-space data comprises cardiac MRI data.

7. The computer-implemented method of claim 6, wherein the reconstructed image comprises a measure of net flow or peak velocity in a heart.

8. A system for MRI imaging, the system comprising:
an MRI machine; and
a controller in operable communication with the MRI machine, wherein the controller comprises a processor and a memory, the memory having computer-executable instructions stored thereon that, when executed by the processor, cause the processor to:
receive k-space data from the MRI machine;
sort the k-space data into a plurality of bins, each of the plurality of bins corresponding to a respective phase of a respiratory cycle;
identify a plurality of outliers in the k-space data; and
reconstruct an image from the k-space data using a compressive sensing (CS) technique configured to remove the plurality of outliers from the k-space data, wherein the CS technique is configured to apply structured sparsity regularization to the plurality of outliers in the k-space data.

9. The system of claim 8, wherein the MRI machine is configured to perform cardiovascular magnetic resonance imaging.

10. The system of claim 8, wherein the CS technique comprises a data fidelity term and a regularization term, wherein the regularization term applies the structured sparsity regularization to the plurality of outliers in the k-space data.

11. The system of claim 8, wherein the CS technique is performed according to
$$\hat{x} = arg {min}_{x , v} \{ \frac{1}{2} {‖ Ax - ( y - v ) ‖}_{2}^{2} + {λ}_{1} {‖ Wx ‖}_{1} + {λ}_{2} {‖ v ‖}_{2 , 1} \} , where \hat{x} = Reconstructed Image y = Collected K Space Data A = Forward Operator W = Sparsifying ( NDW ) Transform v = Rejected Outlier data points and {λ}_{i} = Tuning Parameters .$$

12. The system of claim 8, wherein identifying the plurality of outliers in the k-space data comprises identifying one or more outliers in the k-space data in each of the plurality of bins.

13. The system of claim 8, wherein the reconstructed image is a motion compensated image.

14. The system of claim 8, wherein the k-space data comprises cardiac MRI data.

15. The system of claim 14, wherein the reconstructed image comprises a measure of net flow or peak velocity in a heart.

16. A computer-readable medium having instructions stored therein, wherein execution of the instructions by a processor, causes the processor to:
receive k-space data from an magnetic resonance imaging (MRI) machine;
sort the k-space data into a plurality of bins, each of the plurality of bins corresponding to a respective phase of a respiratory cycle;
identify a plurality of outliers in the k-space data; and
reconstruct an image from the k-space data using a compressive sensing (CS) technique configured to remove the plurality of outliers from the k-space data, wherein the CS technique is configured to apply structured sparsity regularization to the plurality of outliers in the k-space data.

17. The computer-readable medium of claim 16, wherein the CS technique comprises a data fidelity term and a regularization term, wherein the regularization term applies the structured sparsity regularization to the plurality of outliers in the k-space data.

18. The computer-readable medium of claim 16, wherein the CS technique is performed
$$according to \hat{x} = arg {min}_{x , v} \{ \frac{1}{2} {‖ Ax - ( y - v ) ‖}_{2}^{2} + {λ}_{1} {‖ Wx ‖}_{1} + {λ}_{2} {‖ v ‖}_{2 , 1} \} , \\ where \hat{x} = Reconstructed Image y = Collected K Space Data A = Forward Operator W = Sparsifying ( NDW ) Transform v = Rejected Outlier data points and {λ}_{i} = Tuning Parameters .$$

19. The computer-readable medium of claim 16, wherein identifying the plurality of outliers in the k-space data comprises identifying one or more outliers in the k-space data in each of the plurality of bins.

20. The computer-readable medium of claim 16, wherein the reconstructed image is a motion compensated image comprising a measure of net flow or peak velocity in a heart.
